(19) United States
(12) Patent Application Publication
(10) Pub. No.: US 2011/0132439 A1
(43) Pub. Date: Jun. 9, 2011

(54) FULLERENE COMPOUNDS FOR SOLAR CELLS AND PHOTODETECTORS

(75) Inventors: Alex Kwan-yue Jen, Kenmore, WA (US); Yong Zhang, Seattle, WA (US); Hin-Lap Yip, Seattle, WA (US)

(73) Assignee: University of Washington Center for Commercialization, Seattle, WA (US)

(21) Appl. No.: 12/917,923

(22) Filed: Nov. 2, 2010

Related U.S. Application Data
(60) Provisional application No. 61/257,343, filed on Nov. 2, 2009.

(51) Int. Cl. H01L 31/0256; C07C 229/40; C07C 69/003; C07D 209/82; H01B 1/12; H01L 29/78

(52) U.S. Cl. 136/252; 560/43; 560/8; 548/439; 252/500; 257/213; 257/E29.255

(57) ABSTRACT

Amorphous fullerene derivatives and their use in organic electronic devices that include the fullerene derivative as the electron acceptor component in the device's active layer.

### CROSS REFERENCE TO RELATED APPLICATIONS

This application claims priority to and the benefit of co-pending U.S. provisional patent application Ser. No. 61/257,343, filed Nov. 2, 2009, which application is incorporated herein by reference in its entirety.

### STATEMENT OF GOVERNMENT LICENSE RIGHTS

This invention was made with Government support under Grant No. DE-FG36-08-G018024/A000, awarded by the U.S. Department of Energy. The Government has certain rights in this invention.

### FIELD OF THE INVENTION

The present invention relates to fullerene derivatives useful in organic solar cells and photo detectors.

### BACKGROUND OF THE INVENTION

Polymeric solar cells (PSCs) and photodetectors (PDs) have attracted considerable attention in recent years due to their unique advantages of low cost, light weight, solution-based processing and potential application in flexible large area devices ((a) Yu, G.; Gao, J.; Hummelen, J. C.; Wudl, F.; Heeger, A. J., Science 27:1789, 1995; (b) Brabec, C. J.; Sariciftci, N. S.; Hummelen, J. C. Adv. Funct. Mater. 11:15, 2001; (c) Coakley, K. M.; McGehee, M. D., Chem. Mater. 16:4533, 2004; (d) Gnes, S.; Neugebauer, H.; Sariciftci, N. S., Chem. Rev. 107:1324, 2007; (e) Thompson, B. C.; Frechet, J. M. J., Angew. Chem. Int. Ed. 47:58, 2008; (f) Li, Y. F.; Zou, Y. P. Adv. Mater. 20:2952, 2008). Some of the most efficient PSCs and PDs are based on the bulk-heterojunction (BHJ) devices composed of a blend of a conjugated polymer electron donor component and an organic small molecule electron acceptor component. Up to 4-5% of power conversion efficiencies (PCEs) in single-layer PSC devices have been achieved by controlling the morphology of active layer in regioregular poly(3-hexylthiophene) (P3HT) and [6,6]-phenyl C61 butyric acid methyl ester (PC61BM) devices and by developing new low band-gap conjugated polymers blended with fullerene derivatives ((a) Li, G.; Shrotriya, V.; Huang, J.; Yao, Y.; Moriarty, T.; Emery, K.; Yang, Y., Nat. Mater. 4:864, 2005; (b) Peet, J.; Kim, J. Y.; Coates, N. E.; Ma, W. L.; Moses, D.; Heeger, A. J.; Bazan, G. C., Nat. Mater. 6:497, 2007; (c) Ma, W. L.; Yang, C. Y.; Gong, X.; Lee, K.; Heeger, A. J. Adv. Funct. Mater. 15:1617, 2005; (d) Wong, W. Y.; Wang, X. Z.; He, Z.; Djuri\check{s}ić, A. B.; Yip, C. T.; Cheung, K. Y.; Wang, H.; Mak, C. S. K.; Chan, W. K. Nat. Mater. 6:521, 2007). Within the BHJ film, it is critical to control the morphology of the blend to form an interpenetrating network with nano-scale phase separation between the donor and the acceptor at a distance of about 10 nm to maximize exciton dissociation and provide an effective pathway for charge transport and collection ((a) Brabec, C. J.; Sariciftci, N. S.; Hummelen, C. J., Adv. Funct. Mater. 11:15, 2001; (b) Krebs, F. C.; Jørgensen, M.; Norrman, K.; Hagemann, O.; Alstrup, J.; Nielsen, T. D.; Fyenbo, J.; Larsen, K.; Kristensen, J., Sol. Energy Mater. Sol. Cells 93:422, 2009; (c) Krebs, F. C., Sol. Energy Mater. Sol. Cells 93:394, 2009). Currently, PCBMs (including PC61BM and PC71BM) are the most widely used electron acceptor material and gave the highest PCEs with various conjugated polymers ((a) Li, G.; Shrotriya, V.; Huang, J.; Yao, Y.; Moriarty, T.; Emery, K.; Yang, Y., Nat. Mater. 4:864, 2005; (b) Peet, J.; Kim, J. Y.; Coates, N. E.; Ma, W. L.; Moses, D.; Heeger, A. J.; Bazan, G. C., Nat. Mater. 6:497, 2007; (c) Ma, W. L.; Yang, C. Y.; Gong, X.; Lee, K.; Heeger, A. J., Adv. Funct. Mater. 15:1617, 2005). However, PCBMs readily crystallize to form a large aggregation phase (>100 nm), especially on heating. This phenomenon leads to drastic decreases in device efficiency as result of inefficient charge separation and transport because the aggregation phase is much greater than the exciton diffusion length (typically around 10 nm) in the active layer. Furthermore, this also decreases the long-term operation stability of the polymer/PCBM device. In addition to PCBMs, modified PCBMs have also been used in PSCs and organic field effect transistors. However, the device performance of most of these PCBM derivatives is worse than those based on PCBMs, and these devices normally have decreased thermal stability as well.

A need exists for new fullerene derivatives having comparable or improved device efficiency compared to PCBMs and enhanced device stability compared to PCBMs.

### SUMMARY OF THE INVENTION

The invention relates to amorphous fullerene derivatives and their use in organic electronic devices that include the fullerene derivative as the electron acceptor component in the device's active layer.

In one aspect, the present invention provides amorphous fullerene derivatives that are useful as the electron acceptor component in the active layer of PSCs, field-effect transistors, as well as PDs.

In another aspect, the present invention provides devices that include the fullerene derivatives as the electron acceptor component in the active layer. Representative devices include photovoltaic devices such as PSCs, solar windows, and PDs; and field-effect transistors such as PDs.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a cross-sectional view of a photovoltaic device incorporating a representative fullerene derivative of the invention in the active layer.

FIG. 2 is a cross-sectional view of a top contact field-effect transistor device incorporating a representative fullerene derivative of the invention in the active layer.

FIG. 3 is a cross-sectional view of a bottom contact field-effect transistor device incorporating a representative fullerene derivative of the invention in the active layer.

FIG. 4 is a synthesis scheme of TPA-PCBM and MF-PCBM.

FIG. 5 is a plot of DSC traces curves of TPA-PCBM and MF-PCBM and the DSC curve of PCBM was shown as comparison.

FIG. 6 is a plot of cyclic voltammograms of C₆₀, PCBM, TPA-PCBM and MF-PCBM in dichlorobenzene solution.

FIGS. 7A and 7B are plots illustrating transfer characteristics (7A) and output current-voltage characteristics (7B) of PCBM, TPA-PCBM, and MF-PCBM.

FIG. 8 is a plot illustrating current density versus voltage characteristics of PCBM, TPA-PCBM, and MF-PCBM-based BHJ devices under AM1.5 illumination at 100 mW/cm².

FIG. 9 is a plot illustrating PCE change versus annealing time of PCBM, TPA-PCBM, and MF-PCBM-based devices annealed at 150° C.

FIGS. 10A-10C are plots illustrating current density versus voltage characteristics of PCBM (10A), TPA-PCBM (10B), and MF-PCBM (10C)-based OPVs with different annealing times at 150° C.

FIG. 11 summarizes performance of PCBM, TPA-PCBM, and MF-PCBM-based OPVs at optimum processed condition.

FIGS. 12A-12C are optical images of PCBM (12A), TPA-PCBM (12B), and MF-PCBM (12C) devices after annealing at 150° C. for 600 mins.

FIGS. 13A-F are annealing time-dependent optical images of PCBM:P3HT blend films annealed at 150° C.

FIGS. 14A-14C are AFM images of P3HT:PCBM (14A), P3HT:TPA-PCBM (14B), and P3HT:MF-PCBM (14C) films annealed at 150° C. for 600 min.

FIGS. 15A-15C are plots of UV-Vis absorption spectra of P3HT:PCBM (15A), P3HT:TPA-PCBM (15B), and P3HT:MF-PCBM (15C) films annealed at 150° C. for 0 min, 30 min, and 600 min.

### DETAILED DESCRIPTION OF THE INVENTION

The present invention provides a new approach to improve the thermal stability of BHJ photovoltaics and field-effect transistors by employing a new type of amorphous fullerene derivatives as the electron acceptor component in their active layer. The amorphous fullerene derivatives of the invention are obtained by either replacing the planar phenyl ring in PCBM by a bulky electron-rich aromatic functional group or replacing both phenyl ring and butyric acid methyl ester of PCBM by the electron-rich aromatic functional groups. The electron donor properties of functional groups increase the LUMO level of fullerene derivatives, thus increasing the open-circuit voltage of photovoltaics. In addition, after introducing these functional groups, the crystallization tendency of PCBM can be suppressed. Moreover, the same electron donor functional groups can be employed as the building block to prepare the conjugated polymer electron donor component in the BHJ active layer, thus improving the compatibility between the electron donor and acceptor components. Considering the facility of chemical modification of this kind of fullerene derivatives, the invention will provide an excellent approach to develop promising electron acceptor materials for application in PSCs and PDs.

In one aspect, the present invention provides amorphous fullerene derivatives that are useful as the electron acceptor component in the active layer of photovoltaics such as PSCs, solar windows, and PDs, and field-effect transistors such as PDs.

In one embodiment, the amorphous monoadduct fullerene derivatives of the invention have one electron-rich aromatic functional group, as represented by formula (I):

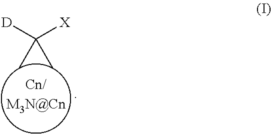

wherein ring Cn is a fullerene core (Cn) or a trimetallic nitride endohedral fullerene core (M₃N@Cn), D is an electron-rich moiety, and X is a nonelectron-deficient moiety. Representative electron-rich moieties include moieties having two or more conjugated phenyl rings, fused benzene rings with at least ten ring carbons, 2,3,4-trisubstitited thiophenes, thiophene oligomers with at least two repeating thiophene units, C4 heteroaryls containing Si or Se, and fused heteroaryls containing S, Si, or Se. Representative X groups include linear or branched alkyl groups having one to 20 carbons, linear or branched ether groups (-L-OR₁), linear or branched ester groups (-L-CO₂R₁), and linear or branched amide groups (-L-CONR₁R₂), wherein L is an alkylene having one to 10 carbons, where R₁ and R₂ are independently selected from hydrogen, an alkyl group having one to 20 carbons, and an aryl group that is unsubstituted or substituted with one or more groups selected from alkyl, alkoxy, alkylamino, and alkylthio.

Representative fullerene cores include C₆₀, C₇₀, C₇₆, C₇₈, C₈₂, C₈₄, and C₉₂ fullerene cores.

Representative metals (M) of the trimetallic nitride endohedral fullerene core include Ga, Sc, Ho, Tb, Gd, Dy, Tm, and Lu.

Representative donors (D) include substituted or unsubstituted triphenyl amine, substituted or unsubstituted tetraphenylbiphenyldiamine, substituted or unsubstituted carbazole, substituted or unsubstituted fluorene, substituted or unsubstituted dibenzosilole, substituted or unsubstituted dibenzothiophene, substituted or unsubstituted dibenthiophene-5,5′-doxide, substituted or unsubstituted naphthalene, and substituted or unsubstituted anthracene, 2,3,4-trisubstitued thiophene, substituted or unsubstituted thiophene oligothiophene, substituted or unsubstituted silole, substituted or unsubstituted selenophene, substituted or unsubstituted thieno[3,2-b]thiophene, substituted or unsubstituted selenolo[3,2-b]selenophene, substituted or unsubstituted cyclopentadithiophene, substituted or unsubstituted silolodithiophene, substituted or unsubstituted stannadithiophene, substituted or unsubstituted dithienopyrrole, substituted or unsubstituted benzo[1,2-b; 4,5-b′]dithiophene, substituted or unsubstituted benzo[1,2-b; 4,3-b′]dithiophene, substituted or unsubstituted phenothiazine, substituted or unsubstituted indenofluorene, substituted or unsubstituted indolocarbazole, substituted or unsubstituted 9-phenylcarbazole, substituted or unsubstituted 10-phenylacridine, substituted or unsubstituted N,N-diphenyl-4-(2-thienyl)-benzenamine.

The donor groups (D) have structures according to the following general formulas, wherein an asterisk (*) in a given structure identifies the point of attachment to the fullerene and that the atom adjacent to the asterisk is missing one hydrogen that would normally be implied by the structure in the absence of asterisk.

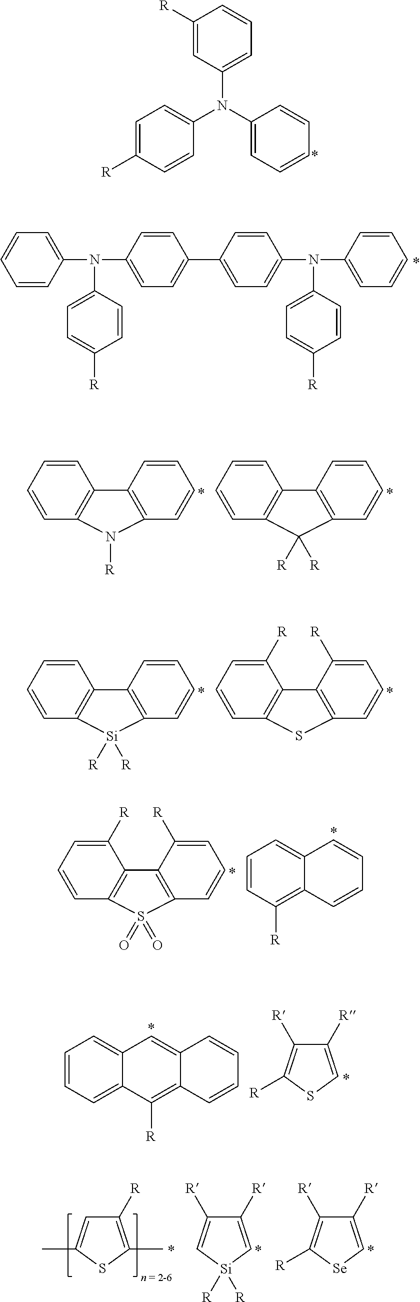

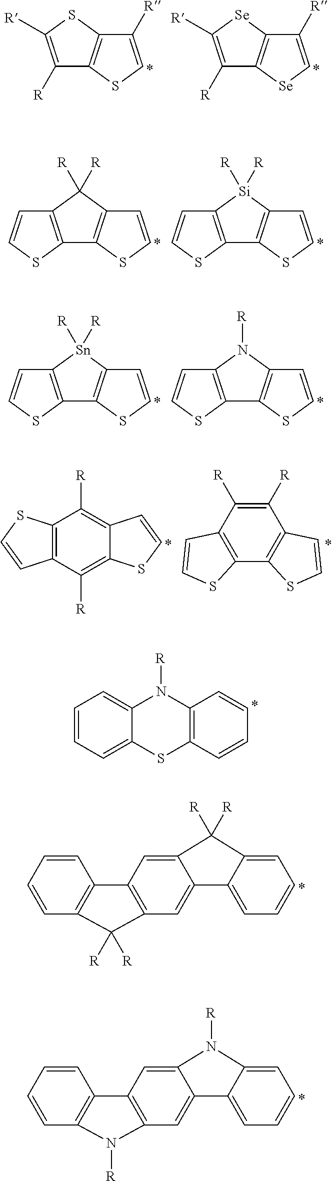

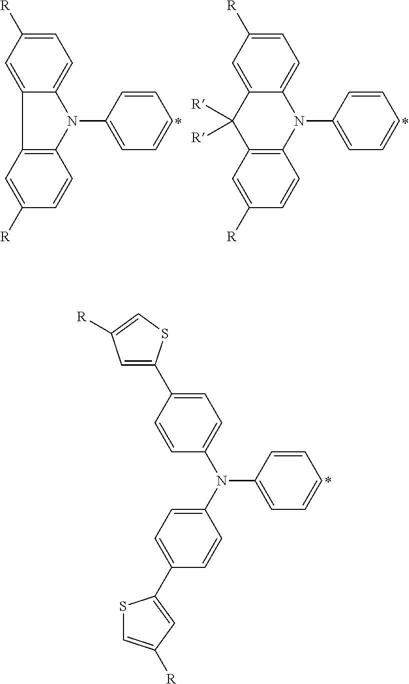

wherein R, R′, and R″ at each occurrence are independently selected from the group consisting of hydrogen, C1-C20 linear or branched alkyl group, C1-C20 linear or branched alkoxy group, C1-C20 linear or branched dialkylamino group, C1-C20 linear or branched alkylthio group.

The following are some examples of fullerene derivatives of the invention:

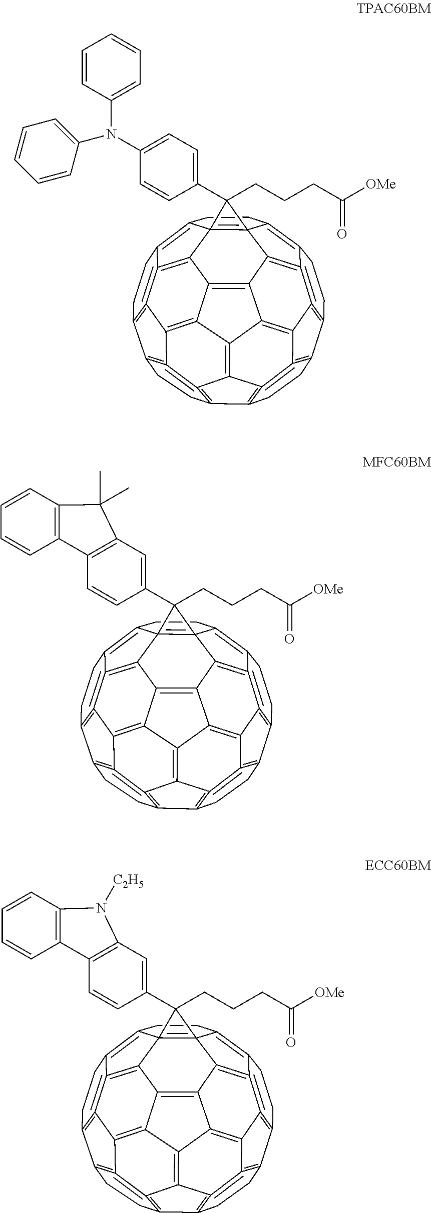

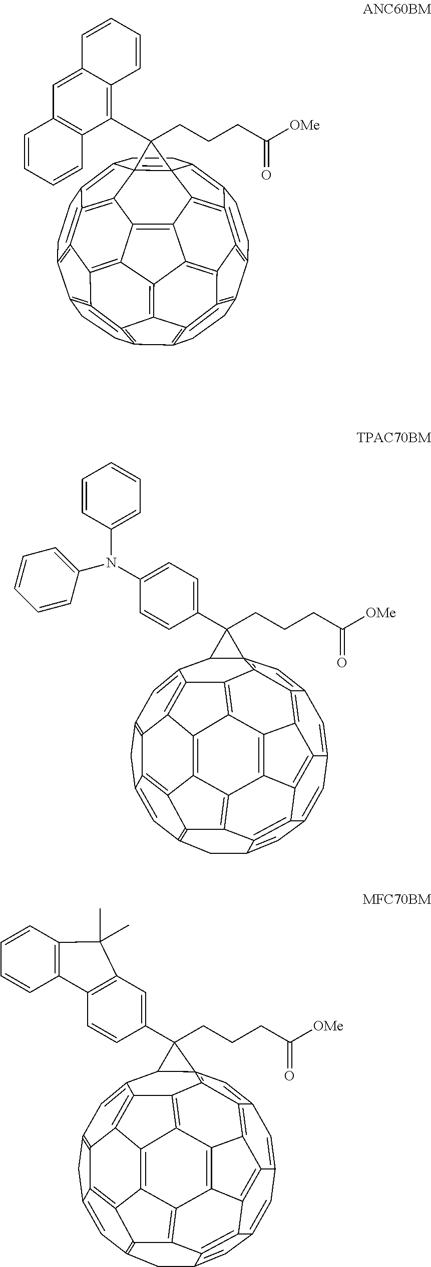

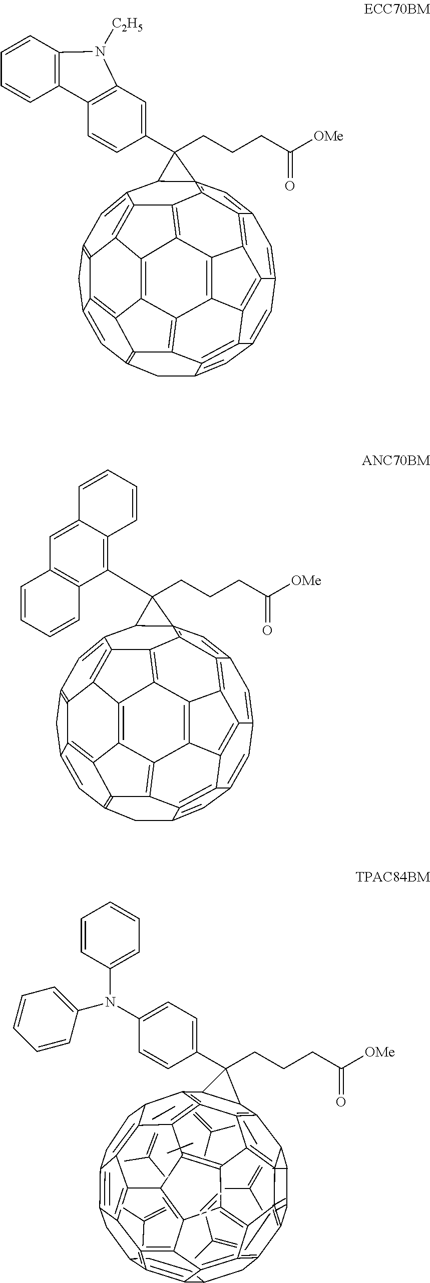

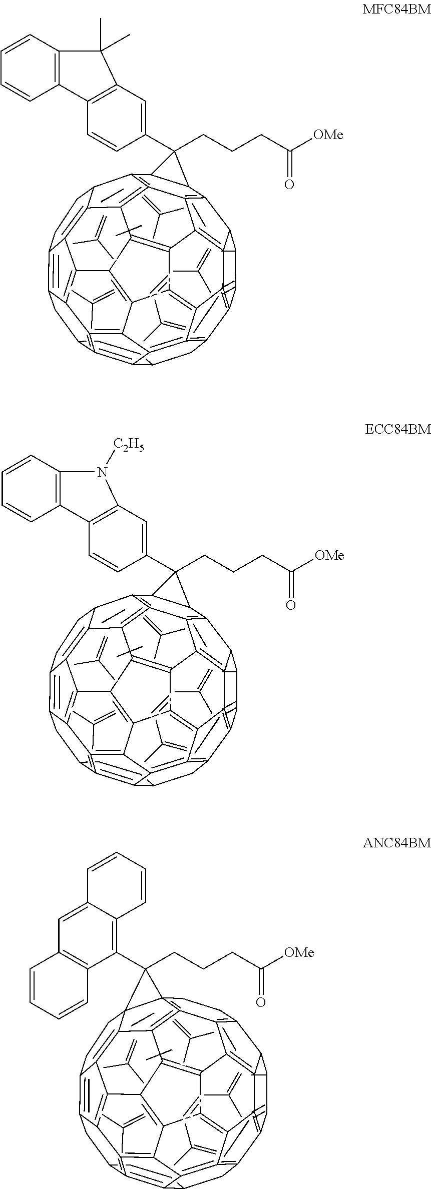

In another embodiment, the amorphous monoadduct fullerene derivatives of the invention have two electron-rich aromatic functional groups, as represented by the following structure (II):

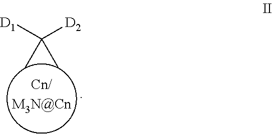

wherein ring Cn is a fullerene core (Cn) or a trimetallic nitride endohedral fullerene core (M₃N@Cn); and D₁ and D₂ are electron-rich moieties. Representative electron-rich moieties include moieties having two or more conjugated phenyl rings, fused benzene rings with at least 10 ring carbons; 2,3,4-trisubstitited thiophenes, thiophene oligomers with at least two repeating thiophene units; C4 heteroaryls containing Si or Se, and fused heteroaryls containing S, Si, or Se.

Representative fullerene cores include C₆₀, C₇₀, C₇₆, C₇₈, C₈₂, C₈₄, and C₉₂ fullerene cores.

Representative metals (M) of the trimetallic nitride endohedral fullerene core include Ga, Sc, Ho, Tb, Gd, Dy, Tm, and Lu.

The donors, D₁ and D₂, at each occurrence are selected from the group consisting of substituted or unsubstituted triphenyl amine, substituted or unsubstituted tetraphenylbiphenyldiamine, substituted or unsubstituted carbazole, substituted or unsubstituted fluorene, substituted or unsubstituted dibenzosilole, substituted or unsubstituted dibenzothiophene, substituted or unsubstituted dibenthiophene-5,5′-doxide, substituted or unsubstituted naphthalene, and substituted or unsubstituted anthracene, 2,3,4-trisubstitued thiophene, substituted or unsubstituted thiophene oligothiophene, substituted or unsubstituted silole, substituted or unsubstituted selenophene, substituted or unsubstituted thieno[3,2-b]thiophene, substituted or unsubstituted selenolo[3,2-b]selenophene, substituted or unsubstituted cyclopentadithiophene, substituted or unsubstituted silolodithiophene, substituted or unsubstituted stannadithiophene, substituted or unsubstituted dithienopyrrole, substituted or unsubstituted benzo[1,2-b; 4,5-b′]dithiophene, substituted or unsubstituted benzo[1,2-b; 4,3-b′]dithiophene, substituted or unsubstituted phenothiazine, substituted or unsubstituted indenofluorene, substituted or unsubstituted indolocarbazole, substituted or unsubstituted 9-phenylcarbazole, substituted or unsubstituted 10-phenylacridine, substituted or unsubstituted N,N-diphenyl-4-(2-thienyl)-benzenamine.

The donor groups (D) have structures according to the following general formulas, wherein an asterisk (*) in a given structure identifies the point of attachment to the fullerene and that the atom adjacent to the asterisk is missing one hydrogen that would normally be implied by the structure in the absence of asterisk.

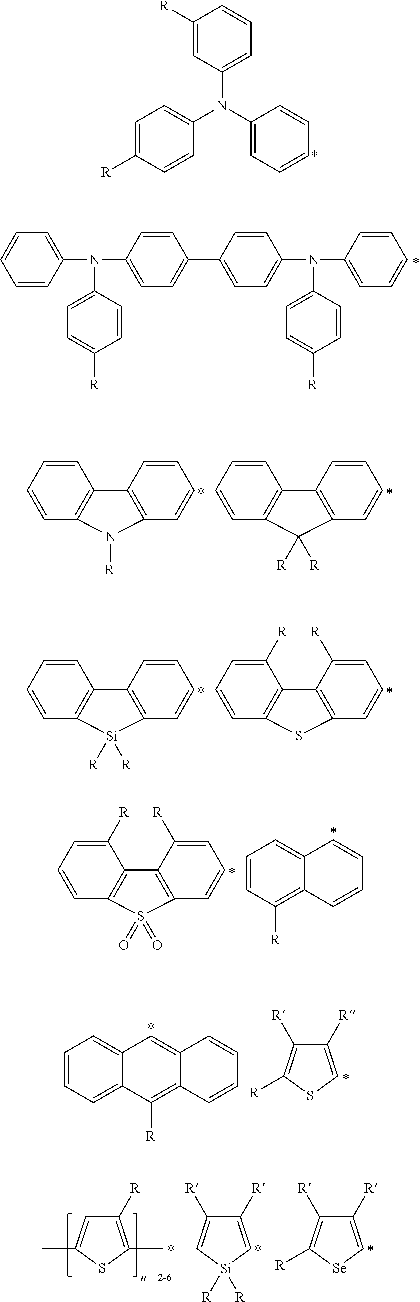

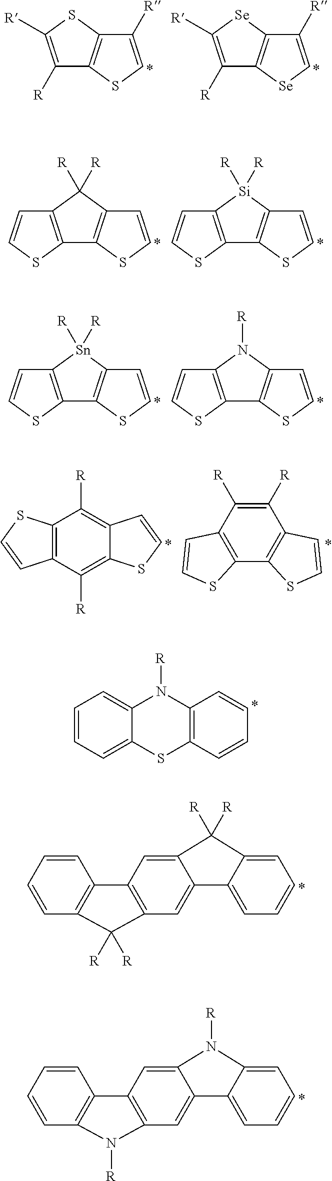

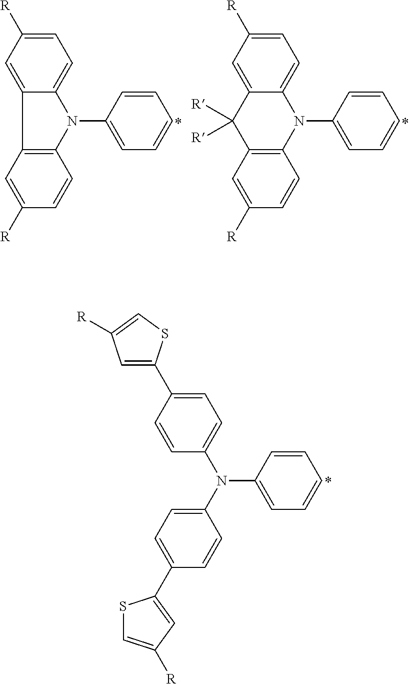

wherein R, R′, and R″ at each occurrence are independently selected from the group consisting of hydrogen, C1-C20 linear or branched alkyl group, C1-C20 linear or branched alkoxy group, C1-C20 linear or branched dialkylamino group, C1-C20 linear or branched alkylthio group.

The novel amorphous fullerene derivatives according to the present invention may be employed as the electron acceptor component in the active layer of photovoltaic devices, field-effect transistors, and photodetectors.

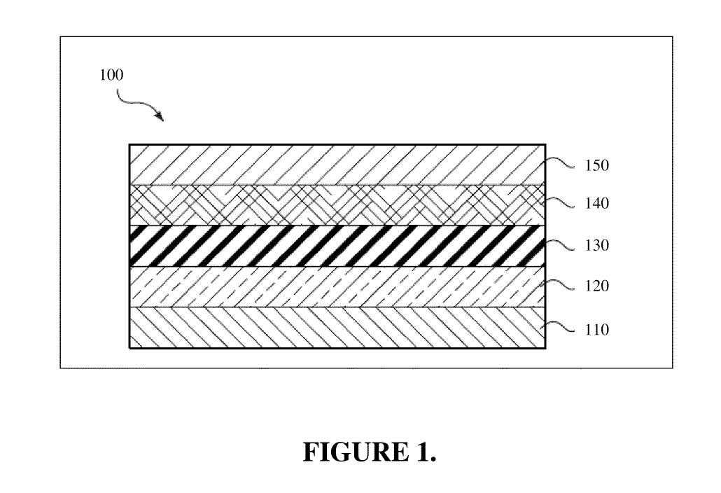

FIG. 1 is a cross-sectional view of a typical heterojunction photovoltaic device in accordance with one embodiment of the invention. Referring to FIG. 1, photovoltaic device 100 includes first electrode 110, first charge-accepting layer 120 formed on first electrode 110, photovoltaic layer 130 formed on first charge-accepting layer 120, second charge-accepting layer 140 formed on photovoltaic layer 130, and second electrode 150 formed on second charge-accepting layer 140. Photovoltaic layer 130 includes, among other active materials, one or more fullerene derivatives of the invention as the electron acceptor component.

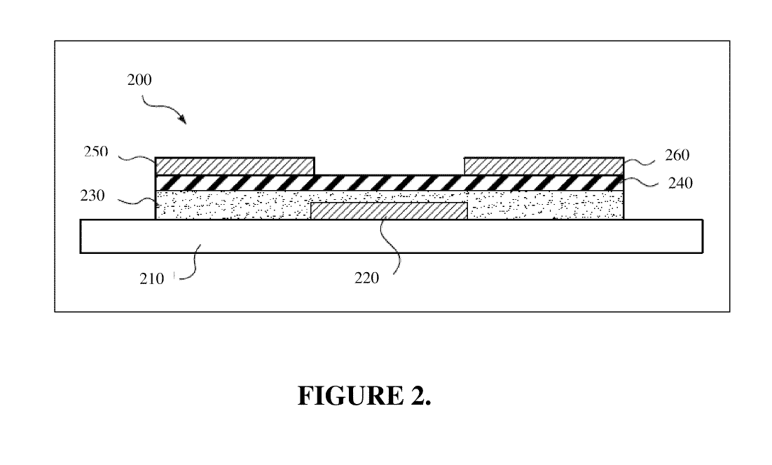

FIG. 2 is a cross-sectional view of a typical top-contact field-effect transistor device in accordance with one embodiment of the invention. Referring to FIG. 2, field-effect transistor device 200 includes substrate 210, gate electrode 220 formed on substrate 210, insulating layer 230 formed on substrate 210 and gate electrode 220, semiconductor layer 240 formed on insulating layer 230, and source electrode 250 and drain electrode 260 formed on semiconductor layer 240. Semiconductor layer 240 includes, among other active materials, one or more fullerene derivatives of the invention as the electron acceptor component.

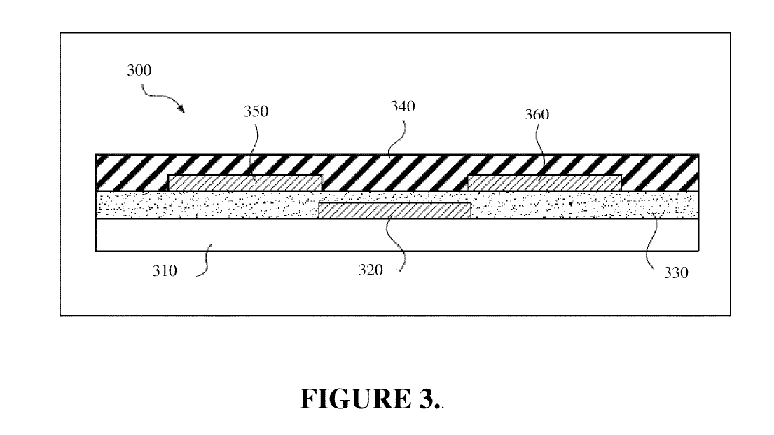

FIG. 3 is a cross-sectional view of a typical bottom-contact field-effect transistor device in accordance with one embodiment of the invention. Referring to FIG. 3, field-effect transistor device 300 includes substrate 310, gate electrode 320 formed on substrate 310, insulating layer 330 formed on substrate 310 and gate electrode 320, source electrode 350 and drain electrode 360 formed on insulating layer 330, and semiconductor layer 340 formed on insulating layer 330, source electrode 350 and drain electrode 360. Semiconductor layer 340 includes, among other active materials, one or more fullerene derivatives of the invention as the electron acceptor component.

The following is a description of the preparation, use, and properties of two fullerene derivatives of the invention: TPA-PCBM and MF-PCBM. PCBM is also mentioned for comparison purpose.

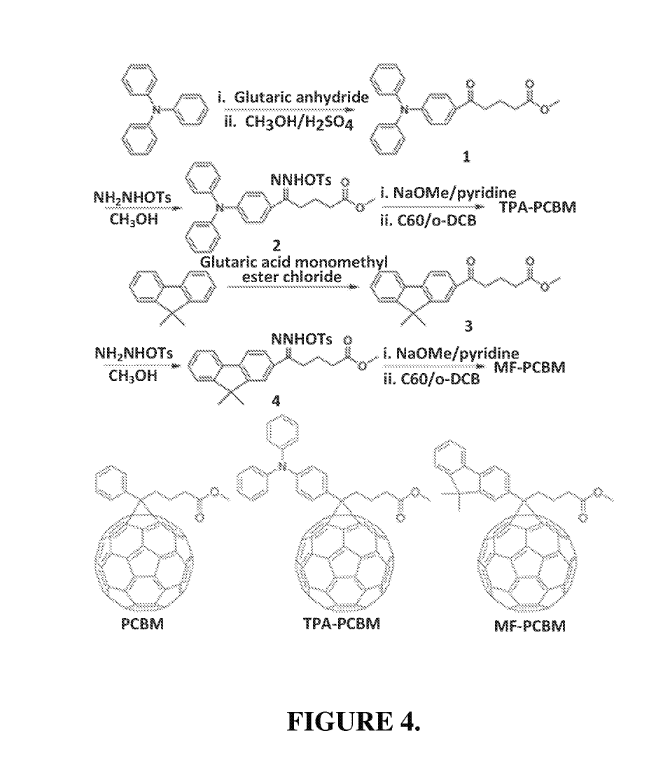

TPA-PCBM and MF-PCBM were obtained by a two-step reaction of the keto-functionalized aromatic methyl butylate with C₆₀. The synthetic route for the fullerenes derivatives is shown in FIG. 4. Compounds 1 and 3 were synthesized by Friedel-Crafts acylation between triphenylamine (TPA) and 9,9-dimethylfluorene (MF) using anhydrous AlCl₃ as catalyst. They were then reacted with p-tosylhydrazide in methanol under refluxing condition to give compounds 2 and 4, respectively. Treatment with sodium methoxide in dried pyridine afforded the diazo compounds, which were used directly to react with C₆₀ in dried o-dichlorobenzene ((a) Hummelen, J. C.; Knight, B. W.; Peq, F. L.; Wudl, F., J. Org. Chem. 60:532, 1995; (b) Zheng, L. P.; Zhou, Q. M.; Deng, X. Y.; Yuan, M.; Yu, G.; Cao, Y., J. Phys. Chem. B. 108:11921, 2004; (c) Yang, C.; Kim, J. Y.; Cho, S.; Lee, J. K.; Heeger, A. J.; Wudl, F., J. Am. Chem. Soc. 130:6444, 2008). The resulting PCBMs were purified through silica column with toluene as eluent to give TPA-PCBM and MF-PCBM with 30-40% yields. As previously reported, the [5,6]-open and [6,6]-closed isomers of [60]methanofullerene could co-exist in the cycloaddition reaction of diazo and [60]fullerene. Therefore, it is necessary to treat these isomers in refluxing o-dichloro-benzene or toluene to form the more stable [6,6]-closed [60]methanofullerene ((a) Hummelen, J. C.; Knight, B. W.; Peq, F. L.; Wudl, F., J. Org. Chem. 60:532, 1995; (b) Zheng, L. P.; Zhou, Q. M.; Deng, X. Y.; Yuan, M.; Yu, G.; Cao, Y., J. Phys. Chem. B. 108:11921, 2004; (c) Yang, C.; Kim, J. Y.; Cho, S.; Lee, J. K.; Heeger, A. J.; Wudl, F., J. Am. Chem. Soc. 130:6444, 2008). The resulting [6,6]-closed [60]methanofullerenes could be easily dissolved in common organic solvents, such as chloroform, toluene, chlorobenzene, and o-dichlorobenzene.

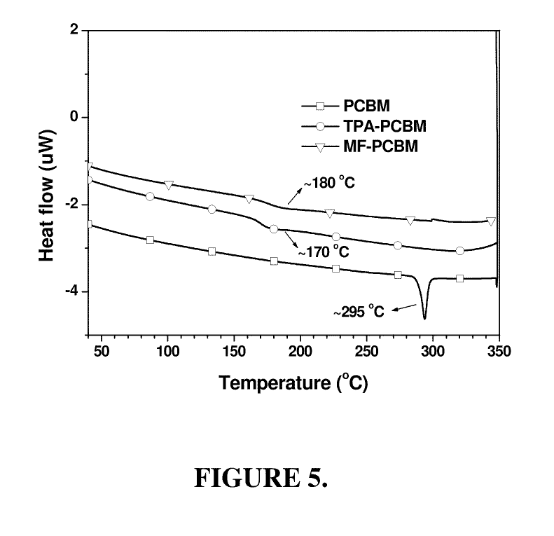

The electrochemical properties of TPA-PCBM and MF-PCBM were studied by cyclic voltammetry in 1,2-dichlorobenzene solution with TBAPF₆ as the supporting electrolyte (shown in FIG. 5). All fullerene derivatives show four quasi-reversible one-electron reduction waves, which are attributed to the fullerene core. The first reduction potential (E₁^{red}) corresponding to the LUMO level of [60]fullerenes is shifted to a more negative value compared to that of C60 due to the decrease of the π-electrons and the release of strain energy after introducing [6,6]methene substitute in C60. In addition, the reduction waves of TPA-PCBM and MF-PCBM are also slightly negative compared to that of PCBM as a result of the stronger electron-donating properties of triphenylamine and 9,9-dimethylfluorene than benzene.

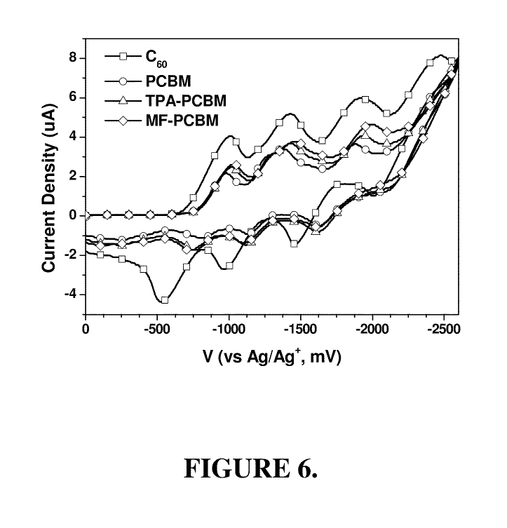

Differential scanning calorimetry (DSC) trace curves of PCBM, TPA-PCBM, and MF-PCBM are shown in FIG. 6. PCBM shows a crystallization peak of 295° C. and there are no other transitions found between 20 and 350° C. However, there are no crystallization transitions found in the curves of TPA-PCBM and MF-PCBM; instead, two glass transitions (T_{g}s) are found at 170 and 180° C. for TPA-PCBM and MF-PCBM, respectively. From the DSC results, the introduction of triphenylamine and dimethylfluorene appear to suppress the crystallization tendency of PCBM. Therefore, it is possible to form a stable electron acceptor phase in TPA-PCBM and MF-PCBM devices to avoid the PCBM aggregation so that the long-term stability of devices could be expected.

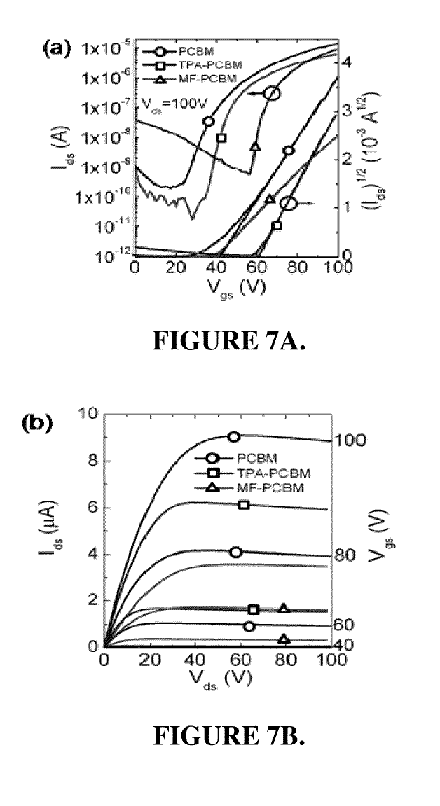

The electron mobility of n-type acceptor is one of the most important factors for high-performance BHJ polymer solar cells. To compare the electron-transporting properties between PCBM and TPA-/MF-PCBMs, n-channel organic field-effect transistors were fabricated. All PCBMs show typical n-type field-effect transistor behavior and the measured saturation field-effect electron mobilities of PCBM, TPA-PCBM and MF-PCBM are 1.6×10⁻², 1.1×10⁻², and 5.4×10⁻³ cm² V⁻¹ s⁻¹, respectively, as shown in FIGS. 7A and 7B. The slight reductions in electron mobilities of TPA-PCBM and MF-PCBM compared to PCBM are attributed to the relatively bulky size of triphenylamine and dimethylfluorene.

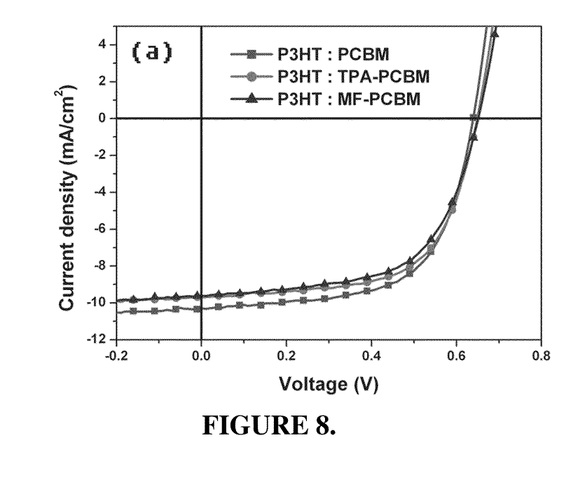

The performance of the P3HT:PCBMs BHJ devices were investigated using an inverted cell structure (ITO/ZnO/C₆₀-SAM/P3HT:PCBMs/PEDOT:PSS/Ag) ((a) Hau, S. K.; Yip, H.-L.; Ma, H.; Jen, A. K.-Y., Appl. Phys. Lett. 93:233304, 2008; (b) Hau, S. K.; Yip, H. L.; Baek, N. S.; Ma, H.; Jen, A. K.-Y., J. Mater. Chem. 18:5113, 2008; (c) Hau, S. K.; Yip, H.-L.; Baek, N. S.; Zou, J.; O'Malley, K.; Jen, A. K.-Y., Appl. Phys. Lett. 92:253301, 2008; (d) Hau, S. K.; Yip, H.-L.; Leong, K.; Jen, A. K.-Y., Org. Electron. 10:719, 2009). This inverted structure using more stable and solution-processed metal as the top electrode can provide better ambient stability and cost advantage than the conventional structure ((a) Hau, S. K.; Yip, H.-L.; Baek, N. S.; Zou, J.; O'Malley, K.; Jen, A. K.-Y., Appl. Phys. Lett. 92:253301, 2008; (b) Hau, S. K.; Yip, H.-L.; Leong, K.; Jen, A. K.-Y., Org. Electron. 10:719, 2009; (c) Krebs, F. C., Sol. Energy Mater. Sol. Cells 92:715, 2008; (d) Krebs, F. C., Sol. Energy Mater. Sol. Cells 93:465, 2009; (e) Krebs, F. C.; Thomann, Y.; Thomann, R.; Andreasen, J. W., Nanotechnology 19:424013, 2008). The optimized device performance for each P3HT/PCBMs system was achieved at a blending ratio of 1:0.7 by weight with 10-30 min annealing at 150° C. FIG. 8 shows the J-V characteristics of P3HT/PCBMs devices under AM 1.5 G illumination with an intensity of 100 mW cm⁻². The power conversion efficiency for TPA-PCBM and MF-PCBM is 4.0% and 3.8%, respectively, which is comparable to that derived from PCBM (4.2%). The TPA-PCBM and MF-PCBM devices have a V_{oc} of 0.65 V, whereas the PCBM device has a V_{oc} of 0.63 V. A 20 mV increase in V_{oc} was observed, which is in agreement with the shift of the LUMO levels as observed in cyclic voltammetry. The short circuit current density (J_{sc}) of TPA-PCBM and MF-PCBM-based devices is 9.9 and 9.8 mA cm⁻², respectively, which is slightly lower than that of PCBM device (10.4 mA cm⁻²) due to lower electron mobilities of the new PCBMs. These results show that TPA-PCBM and MF-PCBM are very promising electron acceptors that give comparable performance to PCBM-derived devices under similar fabrication conditions.

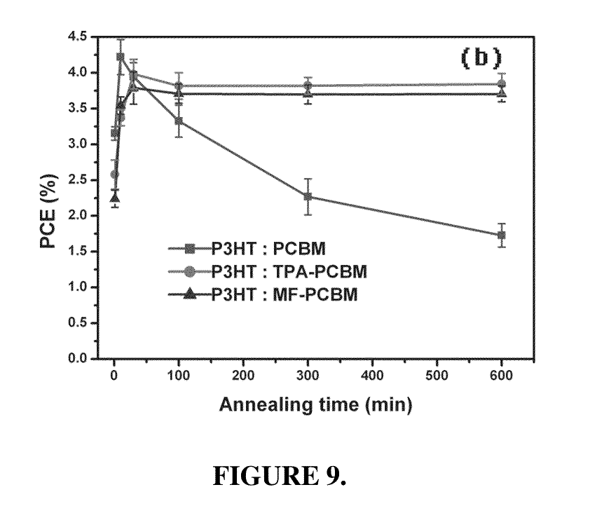

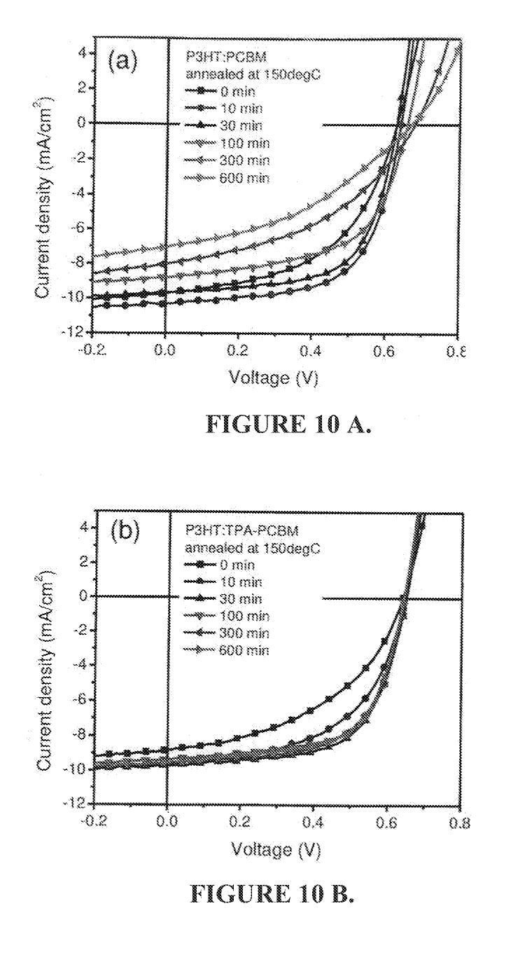

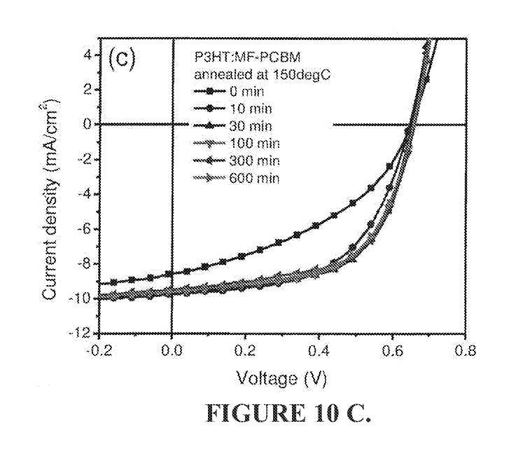

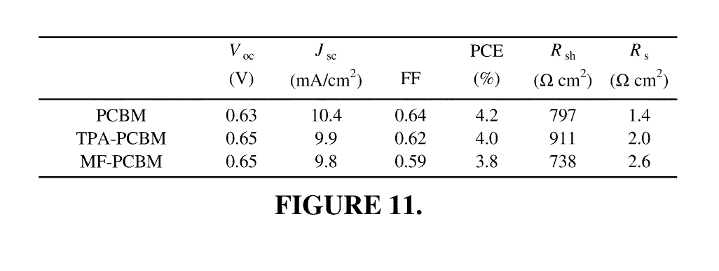

Thermal stability of the photovoltaic devices using these acceptors were examined by annealing the BHJ films at 150° C. for a time period from 10 min to 10 hours. This is a typical temperature for the post-treatment of P3HT:PCBM system. FIG. 9 shows the dependence of PCE on the annealing time of different systems. The highest PCE for the P3HT:PCBM BHJ cell was obtained from the device that was annealed for 10 min. Prolonged annealing results in gradual degradation in device performance with the PCE dropping from 4.2% to 1.8% after annealing for 10 hours. The short circuit current and fill factor also show a gradual decrease with the increase of annealing time. Thermal stability of both TPA-PCBM and MF-PCBM based devices is significantly better than that of PCBM-based device. Even after extended time of annealing (10 hours) there is no obvious loss in device performance (PCE, J_{sc} and FF) with PCE remain at about 4% for both types of devices (FIGS. 10A-10C, FIG. 11).

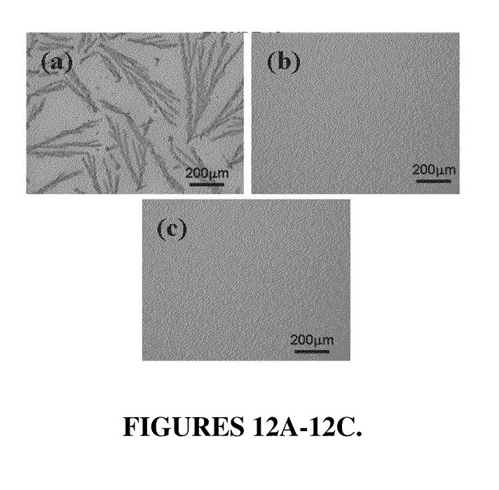

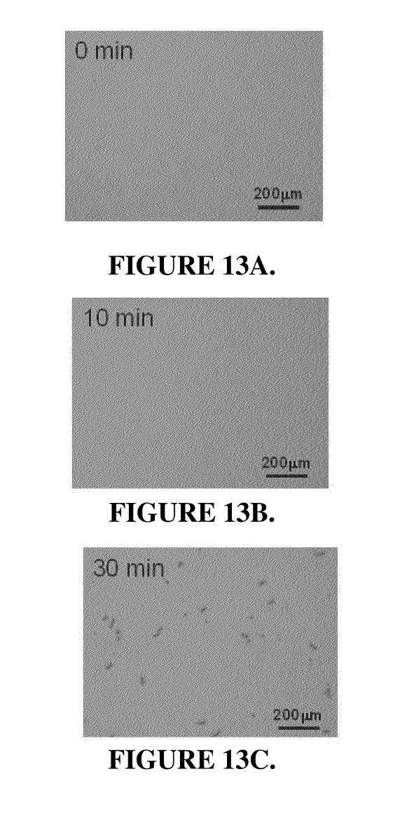

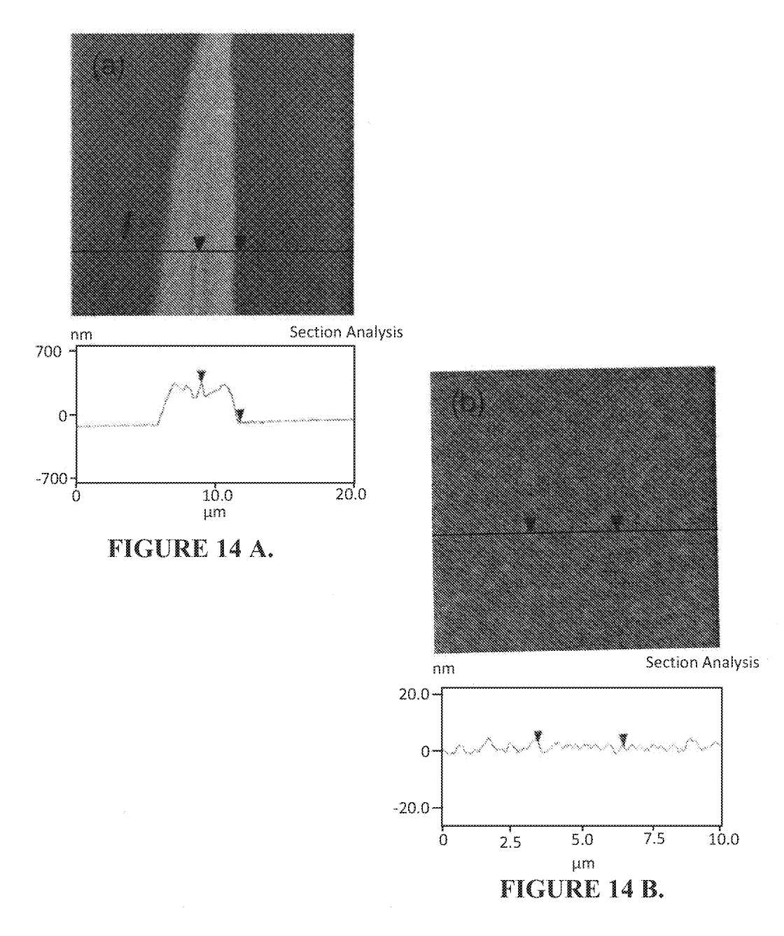

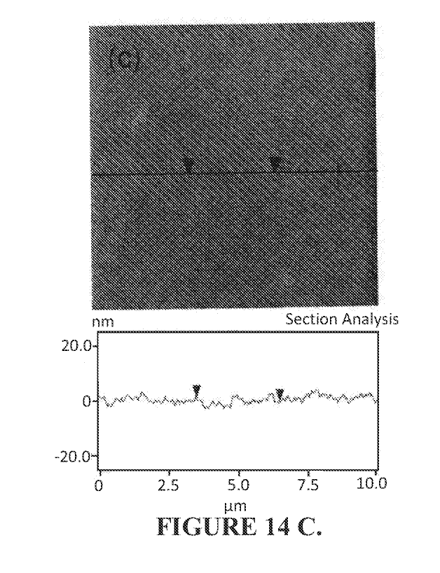

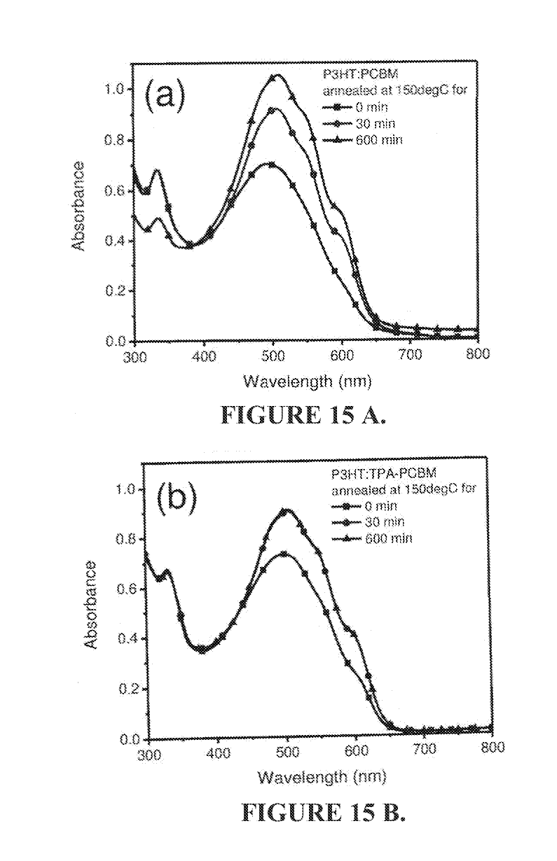

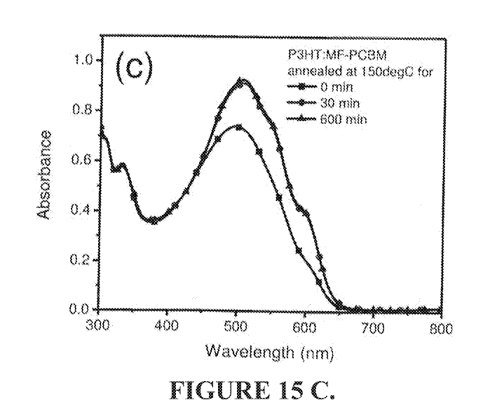

To understand the origin of the improved thermal stability in the amorphous PCBMs-based devices, the effect of thermal annealing on phase segregation in the BHJ films was studied. FIGS. 12A-12C show the optical micrograph of different BHJ films after being annealed at 150° C. for 10 hours. In the case of P3HT:PCBM films, PCBMs aggregated and formed microcrystallites that became larger with longer annealing time. This results in crystal with size up to hundreds of microns in length, tenths of microns in width, and several hundred nanometers in height as revealed by both optical microscopy (FIGS. 13A-13F) and atomic force microscopy (AFM) (FIGS. 14A-14C). Such micron-size crystallization of PCBM causes the reduction of interfacial density between the donor and the acceptor, resulting in decreased excition dissociation efficiency and magnitude of photocurrent. On the contrary, both TPA-PCBM- and MF-PCBM-based BHJ films show no sign of destructive phase segregation even after being annealed for 10 hours. A homogeneous and smooth surface topology was observed by AFM for both blend films with the surface RMS roughness in the range of 1.3-1.5 nm. Furthermore, the absorption spectra of the blend films annealed at different time lengths (0 min, 30 min and 600 min) were also studied (FIGS. 15A-15C). After being annealed for 30 min, three vibronic peaks from the absorption of P3HT (510 nm, 550 nm and 600 nm) become more pronounced for all the blend films, indicating a higher degree of π-π stacking of P3HT chains. Further annealing of TPA-PCBM and MF-PCBM-based films to 600 min does not result in any further change in shape and intensity of the absorption spectrum, which can be correlated well to the enhanced thermal stability of devices. However, the P3HT:PCBM film shows a dramatic decrease in PCBM absorption peak at 335 nm due to the severe segregation of PCBM and a further increase in P3HT vibronic peaks which may be due to improved packing in the P3HT-rich phase (FIGS. 15A-15C).

The present invention provides fullerene derivatives and photovoltaic devices including the fullerene derivative as the electron acceptor component in the active layer.

The following examples are for illustration of the preparation of representative fullerene derivatives of the invention and are not intended to limit the scope of the invention.

### Example 1

### Synthesis of Methyl 4-(N,N-diphenyl)phenyl butyrate (1)

Triphenylamine (5.1 g, 21 mmol) and AlCl₃ (6.0 g, 45 mmol) were dissolved into dry dichloromethane (50 mL) and cooled to 0° C. The glutaric anhydride (2.8 g, 24 mmol) in dry dichloromethane (10 mL) was added slowly into the mixture solution. The mixture was stirred at room temperature for overnight and poured into ice/water, and then, extracted with dichloromethane twice. The combined organic phase was dried over anhydrous MgSO₄, and the solvent was removed under vacuum. The crude triphenylamine-based acid was directed used in next step. The acid crude was dissolved into methanol solution. After adding several drops of concentration H₂SO₄, the methanol solution was heated to reflux for overnight. Then, the mixture was cooled to room temperature and poured into water and extracted with dichloromethane. The organic phase was washed using water for several times and dried over anhydrous MgSO₄. After removing the solvent, the title compound was gotten in the yield of 30% after purifying by silica column. ¹H NMR (CDCl₃, ppm): 7.71 (d, J=9.3 Hz, 2H), 7.24 (m, 4H), 7.06 (m, 6H), 6.89 (d, J=9.0, 2H), 3.60 (s, 3H), 2.86 (t, 2H), 2.33 (t, 2H), 1.96 (t, 2H). ¹³C NMR (CDCl₃, ppm): 197.91, 174.00, 173.56, 152.33, 146.66, 129.79, 126.14, 124.81, 119.89, 51.80, 37.19, 33.43, 33.23, 20.28, 19.87. HRMS (ESI) (M⁺, C₂₄H₂₃NO₃): calcd, 373.1678; found, 373.1662.

### Example 2

### Synthesis of Methyl 4-(N,N-diphenyl)phenyl butyrate p-tosylhydrazone (2)

The compound 1 (0.7 g, 1.9 mmol) and p-toluenesulfonyl hydrazide (0.5 g, 2.7 mmol) were dissolved into methanol with addition of several drops of concentration HCl as catalyst. Then, the mixture solution was reflux for 10 hours. After cooling to room temperature, a white precipitate was collected by filtration and washed using cool methanol twice. The methanol solution was concentrated to around 10 mL and cooled at −4° C. for overnight. The resulted white precipitate was collected by filtration and washed with cool methanol. The combined white solid was dried overnight under vacuum to give the title compound with 74% yield. ¹H NMR (CDCl₃, ppm): 8.99 (s, 1H), 7.91 (d, J=8.4 Hz, 2H), 7.49 (d, J=8.7 Hz, 2H), 7.27 (m, 6H), 7.10 (m, 6H), 6.90 (d, J=8.8 Hz, 2H), 3.82 (s, 3H), 2.59 (t, 2H), 2.42 (s, 3H), 2.32 (t, 2H), 1.67 (m, 2H). ¹³C NMR (CDCl₃, ppm): 174.92, 153.78, 149.31, 147.35, 143.82, 136.24, 129.63, 129.55, 128.17, 127.32, 125.18, 123.75, 122.20, 52.59, 32.31, 25.85, 21.78, 21.22. HRMS (ESI) (M⁺, C₃₁H₃₁N₃O₄S): calcd, 541.2035; found, 541.2022.

### Example 3

### Synthesis of Methyl 2-(9,9-dimethylfluorenyl)butyrate (3)

To a solution of 9,9-dimethylfluorene (3.5 g, 18 mmol) and AlCl₃ (2.8 g, 21 mmol) in dry dichloromethane was added glutaric acid monomethyl ester chloride (3.0 g, 18 mmol) at 0° C. The mixture was stirred at room temperature for overnight. Then, the resulted solution was poured into ice/water, and extracted with dichloromethane. The combined organic phase was dried over anhydrous MgSO₄, and then the solvent was removed under vacuum. The crude product was purified by silica column to give the title compound with 42% yield. ¹H NMR (CDCl₃, ppm): 8.07 (s, 1H), 7.98 (dd, 1H), 7.78 (m, 2H), 7.48 (m, 1H), 7.39 (m, 2H), 3.72 (s, 3H), 3.11 (t, 2H), 2.48 (t, 2H), 2.11 (t, 2H), 1.54 (s, 6H). ¹³C NMR (CDCl₃, ppm): 199.34, 173.98, 154.99, 154.04, 144.28, 138.05, 135.90, 128.75, 127.97, 127.41, 123.00, 122.40, 121.14, 119.98, 51.77, 47.20, 37.75, 33.37, 27.12, 19.73. HRMS (ESI) (M⁺, C₂₁H₂₂O₃): calcd, 322.1569; found, 322.1558.

### Example 4

### Synthesis of Methyl 2-(9,9-dimethylfluorenyl)butyrate p-tosylhydrazone (4)

Compound 3 (1.3 g, 4 mmol) and p-toluenesulfonyl hydrazide (1.5 g, 8 mmol) were dissolved into methanol (15 mL) with addition of several drops of concentration HCl as catalyst. Then, the mixture solution was reflux for 10 hours. After cooling to room temperature, the mixture was poured into water and extracted with dichloromethane. The combined organic phase was dried over MgSO₄. After removing the solvent, the crude product was purified by silica column to give the title compound with 71% yield. ¹H NMR (CDCl₃, ppm): 9.22 (s, 1H), 7.96 (d, J=8.3 Hz, 2H), 7.67-7.45 (m, 5H), 7.32 (m, 4H), 3.84 (s, 3H), 2.67 (t, 2H), 2.43 (s, 3H), 2.37 (m, 2H), 1.75 (m, 2H), 1.50 (s, 6H). ¹³C NMR (CDCl₃, ppm): 174.99, 154.32, 154.16, 153.91, 143.93, 140.89, 138.56, 136.23, 135.32, 129.61, 128.28, 127.94, 127.27, 125.56, 122.86, 120.62, 120.53, 119.96, 52.62, 47.03, 32.29, 27.28, 26.24, 21.78, 21.24. HRMS (ESI) (M⁺, C₂₈H₃₀N₂O₄S): calcd, 490.1926; found, 490.1911.

### Example 5

### Synthesis of [6,6]-[(4-N,N-diphenyl)phenyl]C₆₁-butyric acid methyl ester (TPA-PCBM)

Compound 3 (360 mg, 0.66 mmol) was dissolved into dry pyridine (10 mL) under nitrogen. Then, the sodium methoxide (45 mg) was added quickly under nitrogen, the solution was stirred at room temperature for 20 min. C₆₀ (400 mg, 0.56 mmol) in dichlorobenzene (30 mL) was added in one portion. The resulted purple solution was heated to 70-80° C. and stirred for 48 hours. Then, the solution was heated to reflux and stirred for 24 hours. After cooling to room temperature, the solution was loaded into silica column and pre-eluted with chlorobenzene, then by toluene. The fraction containing TPA-PCBM was collected and concentrated. The concentration solution was poured into methanol solution to give TPA-PCBM with 35% yield. ¹H NMR (CDCl₃, ppm): 7.74 (d, 2H), 7.33 (t, 4H), 7.20 (m, 6H), 7.10 (t, 2H), 3.74 (s, 3H), 2.91 (t, 2H), 2.58 (t, 2H), 2.24 (m, 2H). ¹³C NMR (CDCl₃, ppm): 173.78, 149.19, 148.16, 147.74, 147.61, 146.09, 145.40, 145.36, 145.33, 145.24, 144.99, 144.96, 144.84, 144.68, 144.59, 144.22, 143.98, 143.31, 143.22, 143.18, 143.11, 142.43, 142.31, 141.13, 140.92, 138.17, 137.84, 132.92, 129.62, 125.30, 123.68, 122.01, 80.38, 51.90, 51.69, 34.14, 33.81, 22.66. MALDI-TOF (C₈₄H₂₃NO₂) calcd, 1077.173; found, 1077.136.

### Example 6

### Synthesis of [6,6]-[9,9-dimethylfluorenyl]C₆₁-butyric acid methyl ester (MF-PCBM)

Compound 4 (230 mg, 0.47 mmol) was dissolved into dry pyridine (9 mL) under nitrogen. Then, the sodium methoxide (35 mg) was added quickly under nitrogen, the solution was stirred at room temperature for 20 min. The C60 (281 mg, 0.39 mmol) in dichlorobenzene (33 mL) was added in one portion. The resulted purple solution was heated to 70-80° C. and stirred for 48 hours. Then, the solution was heated to reflux and stirred for 24 hours. After cooling to room temperature, the solution was loaded into silica column and pre-eluted with chlorobenzene, then by toluene. The fraction containing MF-PCBM was collected and concentrated. The concentration solution was poured into methanol solution to give MF-PCBM with 33% yield. ¹H NMR (CDCl₃, ppm): 7.96 (s, 1H), 7.89 (m, 2H), 7.80 (d, 1H), 7.49 (d, 2H), 7.36 (m, 2H), 3.69 (s, 3H), 2.97 (t, 2H), 2.56 (t, 2H), 2.25 (m, 2H), 1.57 (s, 6H). ¹³C NMR (CDCl₃, ppm): 173.67, 154.09, 153.75, 149.17, 148.09, 146.12, 145.38, 145.35, 145.24, 145.19, 144.99, 144.90, 144.84, 144.69, 144.64, 144.20, 143.97, 143.94, 143.19, 143.12, 142.46, 142.33, 142.30, 141.18, 140.91, 139.43, 138.83, 138.26, 137.75, 135.83, 131.27, 129.23, 127.82, 127.34, 126.79, 122.93, 120.45, 120.06, 80.31, 52.55, 51.87, 47.18, 34.13, 33.81, 27.22, 22.71. MALDI-TOF (C₈₁H₂₂O₂) calcd, 1026.162; found, 1026.154.

The ¹H and ¹³C NMR spectra were collected on a Bruker AV 500 spectrometer operating at 500 MHz and 125 MHz in deuterated chloroform solution with tetramethylsilane as reference.

### Example 7

### General Characterization Methods

UV-Vis spectra were studied using a Perkin-Elmer Lambda-9 spectrophotometer. Cyclic voltammetry of different fullerenes was conducted in nitrogen-saturated dichlorobenzene with 0.1 M of tetrabutylammonium hexafluorophosphate using a scan rate of 50 mV s⁻¹. Gold micro-disc, Ag/AgCl and Pt mesh were used as working electrode, reference electrode and counter electrode, respectively. The differential scanning calorimetry (DSC) was performed using DSC2010 (TA instruments) under a heating rate of 20° C. min⁻¹ and a nitrogen flow of 50 mL min⁻¹ AFM images under tapping mode were taken on a Veeco multimode AFM with a Nanoscope III controller.

### Example 8

### Device Fabrication and Characterization

Organic Solar Cells. To fabricate the inverted solar cells, ITO-coated glass substrates (15Ω/□) were cleaned with detergent, de-ionized water, acetone, and isopropyl alcohol. Substrates were then treated with oxygen plasma for 5 min. A thin layer of ZnO nanoparticles (˜50 nm), synthesized using the method described by Beek et. al., was spin-coated onto ITO-coated glass (Beek, W. J. E.; Wienk, M. M.; Kemerink, M.; Yang, X.; Janssen, R. A. J. J. Phys. Chem. B. 2005, 109, 9505). The C₆₀-SAM was then deposited on the ZnO surface using a two-step spin-coating process. First, a 1 mM solution of the molecules in tetrahydrofuran (THF)/chlorobenzene (CB) (1:1 v/v) was spin-coated on the ZnO film. To remove physically absorbed molecules, a second spin-coating using pure THF was applied. Afterward, a CB solution of P3HT (Rieke Metals) and different PCBMs (40 mg/ml) with a weight ratio of (1:0.7) was transferred and spin-coated on the ZnO modified layer to achieve a thickness of (˜200 nm) in a glove box and annealed at 150° C. for different time. After the annealing process, a PEDOT:PSS solution (50 nm) was spin-coated onto the active layer and annealed for 10 min at 120° C. A silver electrode (100 nm) was then vacuum deposited on top to complete the device structure.

The J-V characteristics of the solar cells were tested in air using a Keithley 2400 source measurement unit and an Oriel xenon lamp (450 W) coupled with an AM1.5 filter was used as the light source. The light intensity was calibrated with a calibrated standard silicon solar cell with a KG5 filter which is traced to the National Renewable Energy Laboratory and a light intensity of a 100 mW cm⁻² was used in all the measurements in this study. A physical mask was used to define the device illumination area of 0.0314 cm² to minimize photocurrent generation from the edge of the electrodes. The performance of the OPV was averaged over at least 10 devices for each processed condition. The series resistance (Rₛ) and shunt resistance (Rₛₕ) were calculated from the inverse gradient of the J-V curve at 1 V and 0V, respectively.

Organic Field-Effect Transistors. Top contact organic field-effect transistors (OFETs) were fabricated on heavily n-doped silicon substrates with a 300 nm thick thermally grown SiO₂ dielectric (from Montco Silicon Technologies, Inc.). Before the PCBMs deposition, the substrates were treated with HMDS by vapor phase deposition in a vacuum oven (200 mTorr, 80° C., 5 hrs). The different PCBM films were spin-coated at in a dry argon environment from a 1 wt % chloroform solution to obtain a film thickness of 50 nm. Interdigitated source and drain electrodes (W=9000 μm, L=90 μm, W/L=100) were defined by evaporating a 10 nm Ca followed by 100 nm Al film through a shadow mask from the resistively heated Mo boat at 10⁻⁶ Torr. OFET characterization was carried out in a N₂-filled glovebox using an Agilent 4155B semiconductor parameter S6 analyzer. The field-effect mobility was calculated in the saturation regime from the linear fit of (I_{ds})_{1/2} vs V_{gs}. The threshold voltage (Vₜ) was estimated as the x intercept of the linear section of the plot of (I_{ds})_{1/2} vs V_{gs}. The sub-threshold swing was calculated by taking the inverse of the slope of I_{ds} vs V_{gs} in the region of exponential current increase.

While the preferred embodiment of the invention has been illustrated and described, it will be appreciated that various changes can be made therein without departing from the spirit and scope of the invention.

We claim:

1. A monoadduct fullerene derivative having the formula: 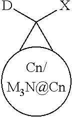
wherein
Cn is a fullerene core or a trimetallic nitride endohedral fullerene core (M₃N@Cn);
D is an electron-rich moiety; and
X is a nonelectron-deficient moiety.

2. The fullerene derivative of claim 1, wherein the fullerene core is selected from the group consisting of C₆₀, C₇₀, C₇₆, C₇₈, C₈₂, C₈₄, and C₉₂.

3. The fullerene derivative of claim 1, wherein the metal in the trimetallic nitride endohedral core is selected from the group consisting of Ga, Sc, Ho, Tb, Gd, Dy, Tm, and Lu.

4. The fullerene derivative of claim 1, wherein D is selected from the group consisting of substituted or unsubstituted triphenyl amine, substituted or unsubstituted tetraphenylbiphenyldiamine, substituted or unsubstituted carbazole, substituted or unsubstituted fluorene, substituted or unsubstituted dibenzosilole, substituted or unsubstituted dibenzothiophene, substituted or unsubstituted dibenthiophene-5,5′-doxide, substituted or unsubstituted naphthalene, and substituted or unsubstituted anthracene, 2,3,4-trisubstitued thiophene, substituted or unsubstituted thiophene oligothiophene, substituted or unsubstituted silole, substituted or unsubstituted selenophene, substituted or unsubstituted thieno[3,2-b]thiophene, substituted or unsubstituted selenolo[3,2-b]selenophene, substituted or unsubstituted cyclopentadithiophene, substituted or unsubstituted silolodithiophene, substituted or unsubstituted stannadithiophene, substituted or unsubstituted dithienopyrrole, substituted or unsubstituted benzo[1,2-b; 4,5-b′]dithiophene, substituted or unsubstituted benzo[1,2-b; 4,3-b′]dithiophene, substituted or unsubstituted phenothiazine, substituted or unsubstituted indenofluorene, substituted or unsubstituted indolocarbazole, substituted or unsubstituted 9-phenylcarbazole, substituted or unsubstituted 10-phenylacridine, substituted or unsubstituted N,N-diphenyl-4-(2-thienyl)-benzenamine.

5. A field-effect transistor device comprising at least one electron donor component and the fullerene derivative of claim 1.

6. A photodetector comprising the device of claim 5.

7. A photovoltaic device comprising the fullerene derivative of claim 1, the photovoltaic device further comprising:
(a) a first electrode;
(b) a first charge-accepting layer disposed on a surface of the first electrode;
(c) an active layer disposed on a surface of the first charge-accepting layer opposite the first electrode, wherein the active layer comprises at least one electron donor component and the fullerene derivative of claim 1;
(d) a second charge-accepting layer disposed on a surface of the active layer opposite the first charge-accepting layer; and
(e) a second electrode disposed on a surface of the second charge-accepting layer opposite the active layer.

8. The photovoltaic device of claim 7, wherein the electron donor component is selected from the group consisting of a polyacetylene, a polyaniline, a polyphenylene, a poly(p-phenylene vinylene), a polythienylvinylene, a polythiophene, a polyporphyrin, a porphyrinic macrocycle, a polymetallocene, a polyisothianaphthalene, a polyphthalocyanine, a discotic liquid crystal polymer, and derivatives and mixtures thereof.

9. A solar cell comprising the photovoltaic device of claim 7.

10. A solar window comprising the photovoltaic device of claim 7.

11. A monoadduct fullerene derivative having the formula: 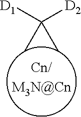
wherein
ring Cn is a fullerene core or a trimetallic nitride endohedral fullerene core (M₃N@Cn); and
D₁ and D₂ are electron-rich moieties.

12. The fullerene derivative of claim 11 wherein the fullerene core is selected from the group consisting of C₆₀, C₇₀, C₇₆, C₇₈, C₈₂, C₈₄, and C₉₂.

13. The fullerene derivative of claim 11, wherein the metal in the trimetallic nitride endohedral core is selected from the group consisting of Ga, Sc, Ho, Tb, Gd, Dy, Tm, and Lu.

14. The fullerene derivative of claim 11, wherein D₁ and D₂ at each occurrence are independently selected from the group consisting of substituted or unsubstituted triphenyl amine, substituted or unsubstituted tetraphenylbiphenyldiamine, substituted or unsubstituted carbazole, substituted or unsubstituted fluorene, substituted or unsubstituted dibenzosilole, substituted or unsubstituted dibenzothiophene, substituted or unsubstituted dibenthiophene-5,5′-doxide, substituted or unsubstituted naphthalene, and substituted or unsubstituted anthracene, 2,3,4-trisubstitued thiophene, substituted or unsubstituted thiophene oligothiophene, substituted or unsubstituted silole, substituted or unsubstituted selenophene, substituted or unsubstituted thieno[3,2-b]thiophene, substituted or unsubstituted selenolo[3,2-b]selenophene, substituted or unsubstituted cyclopentadithiophene, substituted or unsubstituted silolodithiophene, substituted or unsubstituted stannadithiophene, substituted or unsubstituted dithienopyrrole, substituted or unsubstituted benzo[1,2-b; 4,5-b′]dithiophene, substituted or unsubstituted benzo[1,2-b; 4,3-b′]dithiophene, substituted or unsubstituted phenothiazine, substituted or unsubstituted indenofluorene, substituted or unsubstituted indolocarbazole, substituted or unsubstituted 9-phenylcarbazole, substituted or unsubstituted 10-phenylacridine, substituted or unsubstituted N,N-diphenyl-4-(2-thienyl)-benzenamine.

15. A field-effect transistor device comprising at least one electron donor component and the fullerene derivative of claim 11.

16. A photodetector comprising the device of claim 15.

17. A photovoltaic device comprising the fullerene derivative of claim 11, the photovoltaic device further comprising:
(a) a first electrode;
(b) a first charge-accepting layer disposed on a surface of the first electrode;
(c) an active layer disposed on a surface of the first charge-accepting layer opposite the first electrode, wherein the active layer comprises at least one electron donor component and the fullerene derivative of claim 1;
(d) a second charge-accepting layer disposed on a surface of the active layer opposite the first charge-accepting layer; and
(e) a second electrode disposed on a surface of the second charge-accepting layer opposite the active layer.

18. The photovoltaic device of claim 17, wherein the electron donor component is selected from the group consisting of a polyacetylene, a polyaniline, a polyphenylene, a poly(p-phenylene vinylene), a polythienylvinylene, a polythiophene, a polyporphyrin, a porphyrinic macrocycle, a polymetallocene, a polyisothianaphthalene, a polyphthalocyanine, a discotic liquid crystal polymer, and derivatives and mixtures thereof.

19. A solar cell comprising the photovoltaic device of claim 17.

20. A solar window comprising the photovoltaic device of claim 17.
